(19) United States
(12) Patent Application Publication
(10) Pub. No.: US 2023/0215562 A1
(43) Pub. Date: Jul. 6, 2023

(54) SYSTEM AND METHOD FOR INFORMING A USER OF A COVID-19 INFECTION STATUS

(71) Applicant: Specialty Diagnostic (SDI) Laboratories, Inc., Garden Grove, CA (US)

(72) Inventors: Ozman Mohiuddin, Redmond, WA (US); William Henry Haase, Clarksville, TN (US)

(73) Assignee: Specialty Diagnostic (SDI) Laboratories, Inc., Garden Grove, CA (US)

(21) Appl. No.: 17/566,869

(22) Filed: Dec. 31, 2021

(51) Int. Cl. G16H 50/20; G16H 70/60

(52) U.S. Cl. CPC G16H 50/20; G16H 70/60

(57) ABSTRACT

In an aspect a system for informing a user of a COVID-19 infection status is presented. A system includes a computing device configured to receive user data from a user through a diagnostic tool. A computing device is configured to compare user data to an infection criterion. A computing device is configured to determine, as a function of a comparison, a COVID-19 infection status of a user. A computing device is configured to provide a COVD-19 infection status to the user.

### FIELD OF THE INVENTION

The present invention generally relates to the field of COVID-19 testing. In particular, the present invention is directed to a system and method for informing a user of a COVID-19 infection status.

### BACKGROUND

COVID-19 has rapidly spread throughout the world, with millions of people requiring testing, vaccinations, and the like. However, many medical facilities cannot keep up with the demand for COVID-19 testing. As such, a system and method for informing a user of a COVID-19 infection status without the need for a molecular rapid antigen test is presented.

### SUMMARY OF THE DISCLOSURE

In an aspect, a system for informing a user of a COVID-19 infection status is presented. A system includes a computing device configured to receive user data from a user through a diagnostic tool. A computing device is configured to compare user data to an infection criterion. A computing device is configured to determine, as a function of a comparison, a COVID-19 infection status of a user, wherein determining further comprises receiving training data, training an infection status machine learning model as a function of the training data, and determining as a function of the infection status machine learning model and the user data, a COVID-19 infection status of a user. A computing device is configured to provide a COVD-19 infection status to the user.

In yet another non-limiting aspect, a method of informing a user of a COVID-19 infection status using a computing device is presented. A method includes receiving user data from a user through a diagnostic tool. A method includes comparing user data to an infection criterion. A method includes determining, as a function of a comparison, a COVID-19 infection status of a user, wherein determining further comprises receiving training data, training an infection status machine learning model as a function of the training data, and determining as a function of the infection status machine learning model and the user data, a COVID-19 infection status of a user. A method includes providing a COVD-19 infection status to a user.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary embodiment of a system for informing a user of an infection status;

FIG. 2 is an exemplary embodiment of a display of an infection status on a graphical user interface (GUI)

FIG. 3 is an exemplary embodiment of an infection database;

FIG. 4 is an exemplary embodiment of a neural network;

FIG. 5 is an exemplary embodiment of a node of a neural network;

FIG. 6 is an exemplary embodiment of a machine learning model;

FIG. 7 is a flowchart of a method of informing a user of an infection status; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Described herein is a system for informing a user of a COVID-19 infection status. A system may include a computing device configured to receive user data from a user through a diagnostic tool of the diagnostic engine. A computing device may be configured to compare user data to an infection criterion. A computing device may be configured to determine, as a function of a comparison, a COVID-19 infection status of a user. A computing device may be configured to provide a COVD-19 infection status to the user.

Described herein is a method of informing a user of a COVID-19 infection status using a computing device. A method may include receiving user data from a user through a diagnostic tool. A method may include comparing user data to an infection criterion. A method may include determining, as a function of a comparison, a COVID-19 infection status of a user. A method may include providing a COVD-19 infection status to a user.

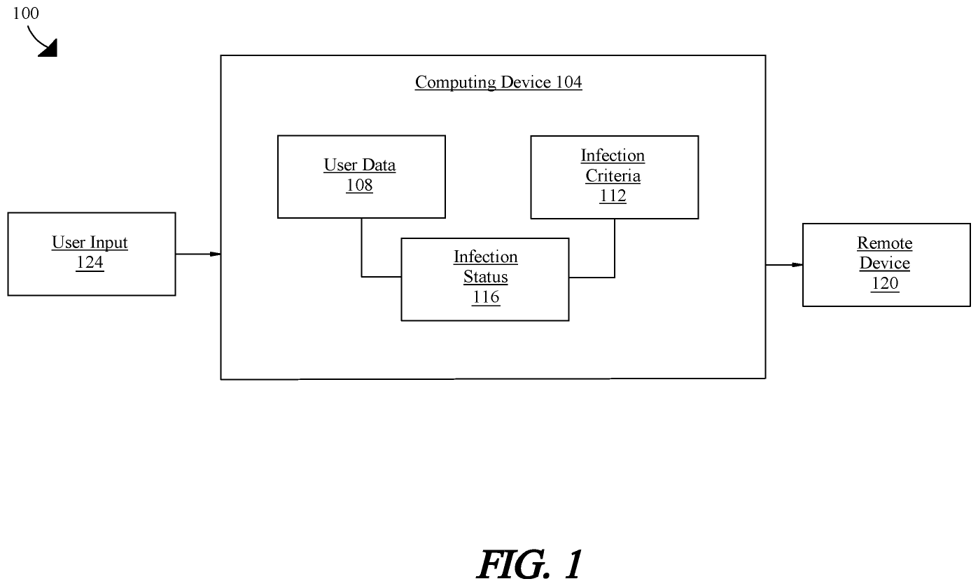

Referring now to FIG. 1, an exemplary embodiment of a system 100 for informing a user of an infection status is illustrated. System 100 includes computing device 104. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device 104.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 may be configured to receive user input 124. “User input” as used in this disclosure is information pertaining to a user's actions. User input 124 may include, but is not limited to, typing on a touch screen, voice inputs, clicking on icons, and the like. User input 124 may be received from a remote computing device. A “remote computing device” as used in this disclosure is a computing device external to a first computing device. A remote computing device may include, but is not limited to, smartphones, laptops, desktops, tablets, and the like. Computing device 104 may generate a digital access link, such as but not limited to, a hyperlink. A digital access link may allow a user to provide user input 124 to computing device 104 from a remote computing device. In some embodiments, computing device 104 may be configured to receive user input 124 over a server. In some embodiments, computing device 104 may receive user input 124 through a mobile application. A mobile application may include software configured to run on a smartphone, tablet, laptop, and the like. A mobile application may generate a graphical user interface configured to receive user input such as, but not limited to, text boxes, icons, scrolling menus, and the like. Computing device 104 may be configured to interact with a user through a mobile application, such as, but not limited to, prompting a user to input user data 108, providing COVID-19 data, and the like. In some embodiments, user input 124 may be received directly on computing device 104. In some embodiments, computing device 104 may generate a diagnostic tool A “diagnostic tool” as used in this disclosure is any instrument capable of extrapolating data from one or more entities. A diagnostic tool may include, but is not limited to, a mobile application, questionnaire, survey, software programs, and the like. Computing device 104 may generate a diagnostic tool that may display a prompt on a remote computing device for a user to provide user input 124. A prompt may include, but is not limited to, a drop-down menu, yes or no checklist, symptom questionnaire, and the like. As a non-limiting example, computing device 104 may generate a diagnostic tool that may prompt for a user to input user data such as demographic information, symptoms, exposure data, underlying medical conditions, and the like. Computing device 104 may receive user data 108 from user input 124 received through a diagnostic tool. “User data” as used in this disclosure is any information pertaining to an individual. User data 108 may include, but is not limited to, age, sex, symptoms, location, family history, potential exposure, vaccination status, travel history, infection history, and the like. Computing device 104 may compare user data 108 with infection criterion 112. “Infection criterion” as used in this disclosure are values constraining an infection determination within a boundary of one or more values. As a non-limiting example, infection criterion may constrain a value of a temperature between 97 to 102 degrees Fahrenheit. Infection criterion 112 may include, but is not limited to, symptoms, exposure, travel history, vaccination status, and the like. In some embodiments, infection criterion 112 may include a weighted criterion. A “weighted criterion” as used in this disclosure is a constraint including a relative importance. Weighted criterion may include a set of constraints having a numerical value adding to a whole number such as 1 or 100. Each constraint may include a numerical value between 0-1, 0-100, and the like. As a non-limiting example, weighted constraints may include high fever (0.4), anosmia (0.3), and/or ageusia (0.3), which together add up to 1. Weighted constraints may be used by computing device 104 in infection criterion 112 to more accurately generate infection status 116. Weighted constraints may be updated through user input, external computing devices, and/or previous iterations of processing. Computing device 104 may update infection criterion 112 through an external database and/or previous iterations of processing. As a non-limiting example, a criterion of infection criterion 112 may include a high fever of at least 102 degrees Fahrenheit. Computing device 104 may determine infected individuals may tend to not have a fever of 102 degrees Fahrenheit and update criterion of infection criterion 112 to include a mild fever of at least 99 degrees Fahrenheit. In some embodiments, Computing device 104 may compare user data 108 to an infection risk threshold. An “infection risk threshold” as used in this disclosure is a maximum or minimum value pertaining to a susceptibility of contracting a disease. An infection risk threshold may include, but is not limited to, geographical data, demographic data, travel data, contact data, and the like. An infection risk threshold may be determined by use of a machine learning model. As a non-limiting example, computing device 104 may compare user data 108 to an infection risk threshold and determine that a user may have a weakened immune system and recent contact with an infected person and therefore is at high risk for contracting an infection. Computing device 104 may generate a risk profile for a user as a function of user data 108. A “risk profile” as used in this disclosure is a set of data pertaining to an individual's susceptibility to a disease. A risk profile may include data pertaining to a user's susceptibility to contracting an infection. As a non-limiting example, a risk profile of a user may show that a user is at low risk of contracting COVID-19 due to the user's vaccinations, young age, and no underlying health conditions. In some embodiments, computing device 104 may communicate with an infection expert database to determine infection status 116. An infection expert database may include any database suitable for use as in infection expert database. Computing device 104 may compare user data 108 with infection expert data of an infection expert database to determine infection status 116. In some embodiments, computing device 104 may update an infection status 116 of a user as a function of another infection status 116 of another user. As a non-limiting example, a first user may have a negative COVID-19 infection status. However, a second user may have a positive COVID-19 infection status, which may cause a first user's infection status to change to positive for COVID-19 based on recent contact, exposure, similar symptoms, and the like. In some embodiments, computing device 104 may be configured to alert a user of an infection risk as a function of a proximity criteria. A “proximity criteria” as used in this disclosure is a constraint of an infection risk pertaining to geographical data. Proximity criteria may include, but is not limited to, feet, meters, miles, GPS coordinates, radiuses, and the like. Computing device 104 may determine a high infection risk for a user approaching an area with a high infection rate, which may meet a proximity criteria and trigger computing device 104 to send an alert to the user.

Still referring to FIG. 1, in some embodiments, computing device 104 may be configured to be run on a server. Computing device 104 may utilize an infection status machine learning model. An infection status machine learning model may be trained with training data correlating user data and/or infection criteria to infection statuses. Training data may be received from user input, an external computing device, and/or previous iterations of processing. An infection status machine learning model may be configured to input user data and output an infection status. In some embodiments, computing device 104 may be configured to analyze user data 108 and infection criterion 112 to produce infection status 116. An “infection status” as used in this disclosure is an indicator of whether an individual is carrying a disease. Infection status 116 may include statuses such as, but not limited to, “infected”, “not infected”, “prone to infection”, “not prone to infection”, “high risk” “low risk” and the like. Infection status 116 may include a positive and/or negative status of a user having a disease such as COVID-19. Infection status 116 may include a suspected positive and/or negative status of a user having a disease such as but not limited to any infectious disease, any deficiency disease, any hereditary disease, and/or any physiological disease. Infection status 116 may include any disease, injury, disability, disorder, syndrome, infection, insolated symptoms, deviant behaviors, and/or atypical variations of structure and function. In some embodiments, infection status 116 may include a coronavirus infection status such as a COVID-19 infection status. In some embodiments infection status 116 may include a viral infection status, a bacterial infection status, a fungi infection status, and/or a parasitic infection status. A “COVID-19 infection status” as used in this disclosure is an indicator pertaining to an individual's contraction of COVID-19. In some embodiments, infection status 116 may be provided to remote device 120. Infection status 116 may be displayed through a graphical user interface (GUI) of remote device 120. In some embodiments, computing device 104 may communicate medical consultation with remote device 120 based on infection status 116. As a non-limiting example, infection status 116 may indicate a user has COVID-19. Computing device 104 may determine a user has severe underlying conditions and should consult with a physician. Computing device 104 may display a recommended consultation on remote device 120. In some embodiments, computing device 104 may send alerts to one or more remote devices as a function of infection status 116. As a non-limiting example, computing device 104 may determine an individual may be infected with COVID-19. Computing device 104 may send alerts to individuals in contact and/or in recent contact with an infected individual informing the contacts of the infection status of the infected individual. Computing device 104 may be configured to rapidly determine infection status 116 of a user, such as within 5 minutes, 2 minutes, and the like.

Still referring to FIG. 1, in some embodiments computing device 104 may use a fuzzy inference system. “Fuzzy inference” is the process of formulating a mapping from a given input to an output using fuzzy logic. “Fuzzy logic” is a form of many-valued logic in which the truth value of variables may be any real number between 0 and 1. Fuzzy logic may be employed to handle the concept of partial truth, where the truth value may range between completely true and completely false. The mapping of a given input to an output using fuzzy logic may provide a basis from which decisions may be made and/or patterns discerned. A first fuzzy set may be represented, without limitation, according to a first membership function representing a probability that an input falling on a first range of values is a member of the first fuzzy set, where the first membership function has values on a range of probabilities such as without limitation the interval [0,1], and an area beneath the first membership function may represent a set of values within the first fuzzy set. A first membership function may include any suitable function mapping a first range to a probability interval, including without limitation a triangular function defined by two linear elements such as line segments or planes that intersect at or below the top of the probability interval.

Still referring to FIG. 1, a first fuzzy set may represent any value or combination of values as described above, user data, infection criteria, and/or any combination of the above. A second fuzzy set, which may represent any value which may be represented by first fuzzy set, may be defined by a second membership function on a second range; second range may be identical and/or overlap with first range and/or may be combined with first range via Cartesian product or the like to generate a mapping permitting evaluation overlap of first fuzzy set and second fuzzy set. Where first fuzzy set and second fuzzy set have a region that overlaps, first membership function and second membership function may intersect at a point representing a probability, as defined on probability interval, of a match between first fuzzy set and second fuzzy set. Alternatively or additionally, a single value of first and/or second fuzzy set may be located at a locus on a first range and/or a second range, where a probability of membership may be taken by evaluation of a first membership function and/or a second membership function at that range point. A probability may be compared to a threshold to determine whether a positive match is indicated. A threshold may, in a non-limiting example, represent a degree of match between a first fuzzy set and a second fuzzy set, and/or single values therein with each other or with either set, which is sufficient for purposes of the matching process. In some embodiments, there may be multiple thresholds. Each threshold may be established by one or more user inputs. Alternatively or additionally, each threshold may be tuned by a machine-learning and/or statistical process, for instance and without limitation as described in further detail below.

Still referring to FIG. 1, an inference engine may be implemented according to input and/or output membership functions and/or linguistic variables. For instance, a first linguistic variable may represent a first measurable value pertaining to a datum of user data, such as fevers, travel history, exposure, and the like thereof, while a second membership function may indicate a degree of COVID-19 infection status. Continuing the example, an output linguistic variable may represent, without limitation, a score value and/or infection criterion. An inference engine may combine rules, such as any, semantic language, infection criterion, infection risk threshold ranges, and the like thereof. An inference engine may further combine rules that indicate how likely a positive or negative COVID-19 infection status is based on user data—the degree to which a given input function membership matches a given rule may be determined by a triangular norm or “T-norm” of the rule or output membership function with the input membership function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥,” such as max(a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

Still referring to FIG. 1, computing device 104 may use a fuzzy inference system to determine a plurality of outputs based on a plurality of inputs. A plurality of outputs may include, but is not limited to, positive for COVID-19, negative for COVID-19, high risk for COVID-19, low risk for COVID 19, high exposure to COVID-19, low exposure to COVID-19, and the like. As a non-limiting example, computing device 104 may determine a high fever of a user from user data 108 along with a recent travel of the user to a high-infection risk area. computing device 104 may determine inputs as “high fever” and “high exposure risk” and output “high probability of COVID- 19 infection”. In another non-limiting example, computing device 104 may determine from user data 108 that a user is not vaccinated and over the age of 60. computing device 104 may determine inputs as “not vaccinated” and “elderly” and output “high-risk of contracting COVID-19”. computing device 104 may use a fuzzy inference system to determine infection status 116 and improve accuracy of infection status 116. computing device 104 may use a fuzzy inference system to generate a preliminary diagnosis of a user by comparing user data 108 to infection criterion 112. As a non-limiting example, computing device 104 may determine from user data 108 that a user may be experiencing anosmia and/or ageusia. Anosmia and/or ageusia may be criterion of infection criterion 112. computing device 104 may determine inputs as “symptomatic” and “meets infection criterion” and output “high likelihood of COVID-19 infection”. In another non-limiting example, computing device 104 may determine user data 108 meets 4 out of 5 criterion of infection criterion 112, such as recent contact, high exposure, fever, and cough. computing device 104 may determine an input as “meets majority of infection criterion” and output “high likelihood of COVID-19 infection”.

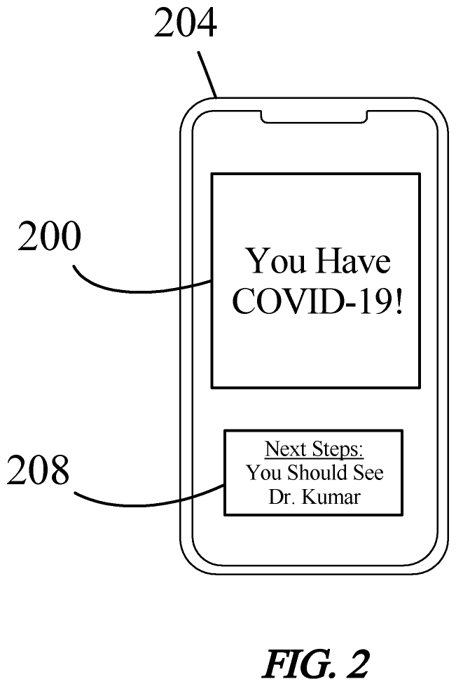

Referring now to FIG. 2, an exemplary embodiment of an infection status 200 on a remote device 204 is shown. Remote device 204 may include, but is not limited to, smartphones, tablets, laptops, desktops, and the like. Infection status 200 may include a positive or negative indicator of a user having COVID-19. Infection status 200 may include but is not limited to, confidence scores of infection status 200, recent contacts, recent exposures, underlying medical risks, and the like. In some embodiments, medical consultation 208 may be displayed on remote device 204. Medical consultation may include, but is not limited to, medical care facility selection, medical specialist selection, medical services, and the like. As a non-limiting example, a user may be shown infection status 204 as a positive indicator of COVID-19, and may also be shown medical consultation 208 indicating that the user should see Dr. Kumar.

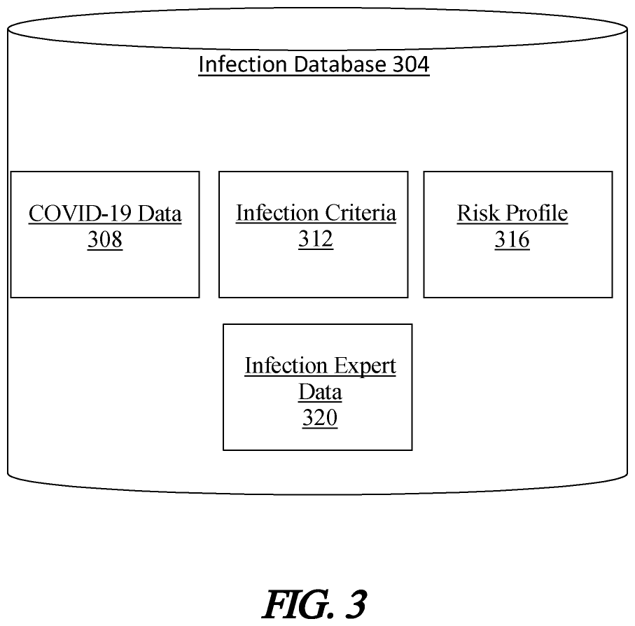

Referring now to FIG. 3, an exemplary embodiment of infection database 304 is shown. Infection database 304 may be implemented, without limitation, as a relational database, a key-value retrieval database such as a NOSQL database, or any other format or structure for use as a database that a person skilled in the art would recognize as suitable upon review of the entirety of this disclosure. Infection database 304 may alternatively or additionally be implemented using a distributed data storage protocol and/or data structure, such as a distributed hash table or the like. Infection database 304 may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Still referring to FIG. 3, computing device 104 may communicate with infection database 304 to improve accuracy in diagnosing infection status 116. Infection database 304 may include COVID-19 data 308. COVID-19 Data 308 may include data such as, but not limited to, symptoms, infection trends, variant data, high spread areas, low spread areas, and the like.

Still referring to FIG. 3, infection database 304 may include infection criterion 312. Infection criterion 312 may include criterion such as, but not limited to, symptoms, recent contacts, exposure thresholds, travel history, and the like.

Still referring to FIG. 3, infection database 304 may include risk profile 316. Risk profile 316 may include data of a user relating to a likelihood of contracting COVID-19. Risk profile 316 may include data such as, but not limited to, underlying medical conditions, demographic data, travel history, contact history, and the like.

Still referring to FIG. 3, infection database 304 may include infection expert data 320. Infection expert data 320 may include data received from an infection expert. An “infection expert” as used in this disclosure is an individual knowledgeable in diseases. In some embodiments, an infection expert may include a member of the Center for Disease Control (CDC). Infection expert data 320 may include data such as new variants of COVID-19, new symptoms, trends of infection, infection spreads, vaccination data, and the like. Infection database 304 may update COVID-19 data 308, infection criterion 312, and/or risk profile 316 based on data from Infection expert data 320.

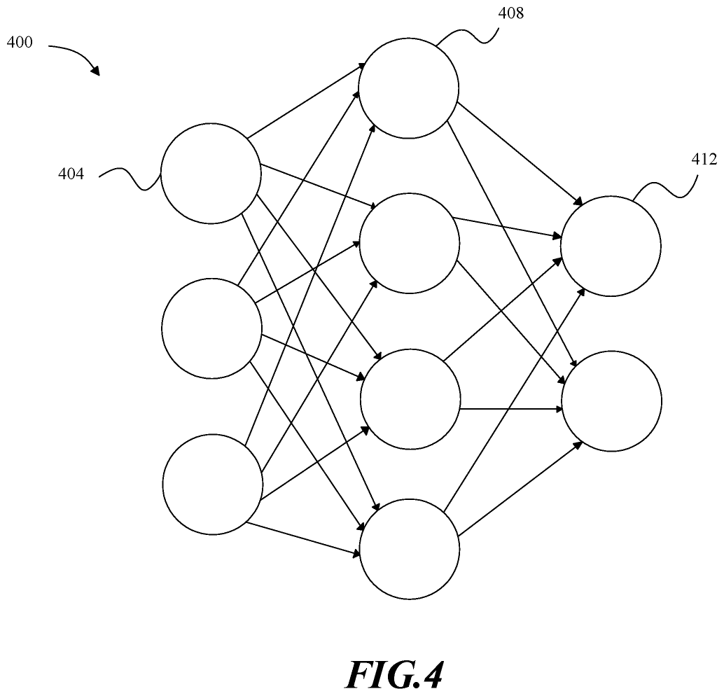

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.”

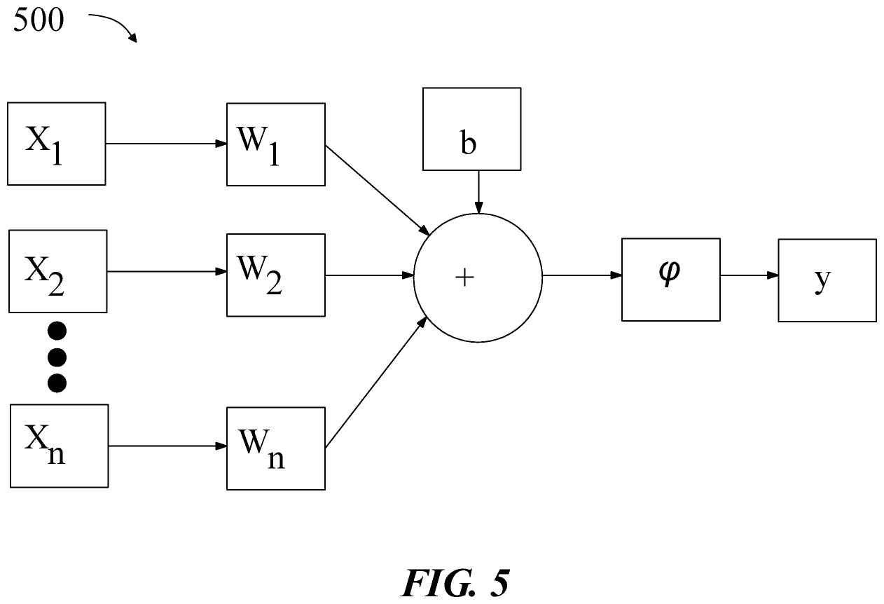

Referring now to FIG. 5, an exemplary embodiment of a node of a neural network is illustrated. A node may include, without limitation a plurality of inputs xi that may receive numerical values from inputs to a neural network containing the node and/or from other nodes. Node may perform a weighted sum of inputs using weights wᵢ that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

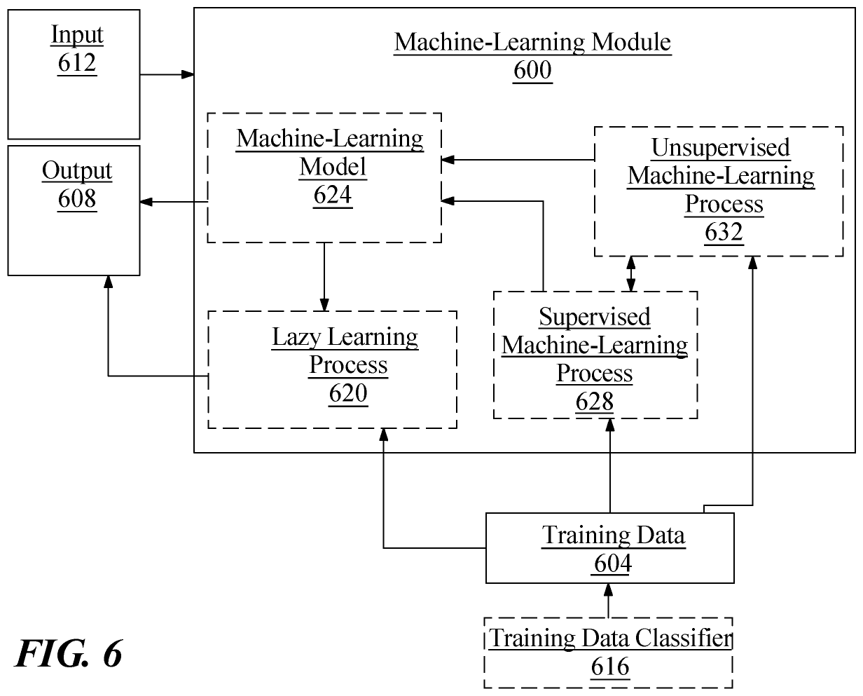

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 604 may be provided in fixed-length formats, formats linking positions of data to categories such as comma-separated value (CSV) formats and/or self-describing formats such as extensible markup language (XML),

JavaScript Object Notation (JSON), or the like, enabling processes or devices to detect categories of data.

Alternatively or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non- limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure. As a non-limiting illustrative example inputs may include user data and/or infection criteria and outputs may include infection statuses.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 616 may classify elements of training data to COVID-19 symptoms, risk factors, demographic data, contraction factors, and the like.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine- learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include user data as described above as inputs, infection statuses as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminate analysis. Machine-learning algorithms may include kernel ridge regression. Machine- learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine- learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

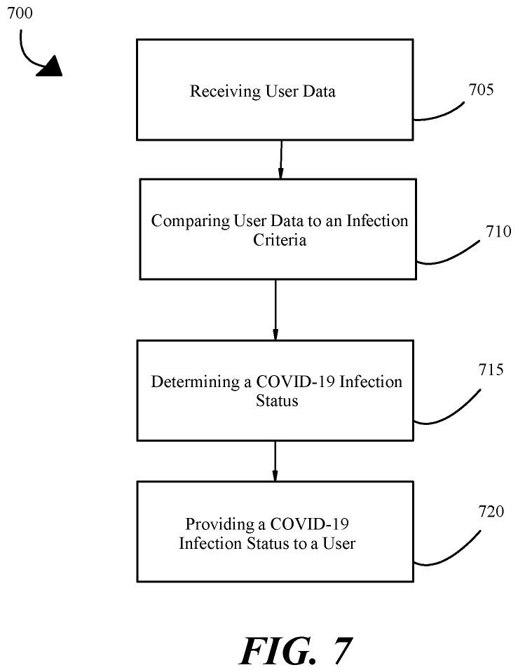

Referring now to FIG. 7, method 700 of informing a user of an infection status is presented. At step 705, method 700 includes receiving user data. Receiving user data may include receiving user data through a diagnostic tool from a remote computing device. User data may include, but is not limited to, demographic data, travel data, exposure data, contact data, and the like. This step may be implemented as described above in FIGS. 1-6.

Still referring to FIG. 7, at step 710, method 700 includes comparing user data to an infection criterion. In some embodiments, a comparison may include using an infection status machine learning model. A comparing may include using a fuzzy inference system. This step may be implemented as described above in FIGS. 1-6.

Still referring to FIG. 7, at step 715, method 700 includes determining a COVID-19 infection status. A COVID-19 infection status may include, but is not limited to, positive, negative, high-risk, low-risk, and the like. This step may be implemented as described above in FIGS. 1-6.

Still referring to FIG. 7, at step 720, method 700 providing a COVID-19 infection status to a user. A COVID-19 infection status may be presented to a user on a remote computing device, such as through a GUI of a smartphone. In some embodiments, providing a COVID-19 infection status to a user may include providing medical consultation to a user. This step may be implemented as described above in FIGS. 1-6.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

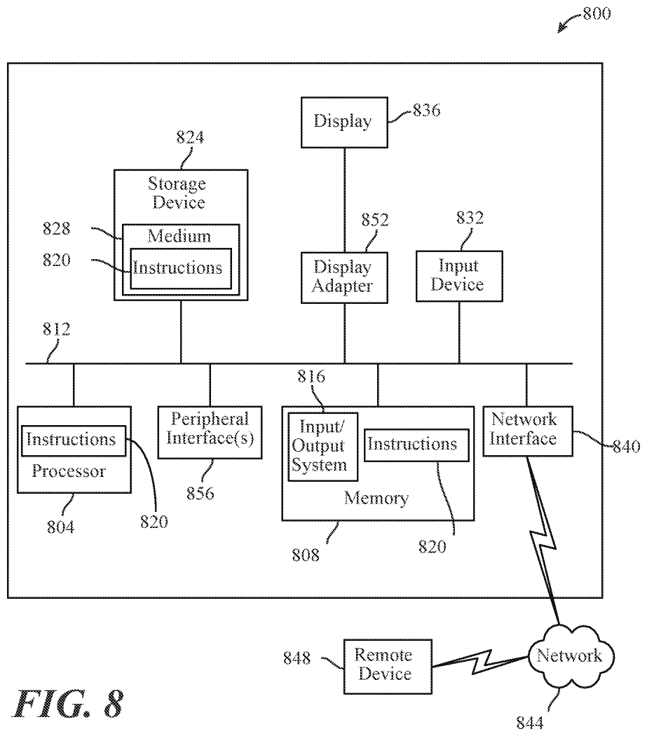

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Still referring to FIG. 8, processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Still referring to FIG. 8, memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Still referring to FIG. 8, computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Still referring to FIG. 8, computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

Still referring to FIG. 8, a user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Still referring to FIG. 8, computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for informing a user of a COVID-19 infection status, comprising:
a computing device configured to:
receive user data, comprising user symptoms, from a user through a diagnostic tool;
compare the user data to an infection criterion, wherein the infection criterion comprises a weighted criterion configured to be updated as a function of previously generated infection statuses;
determine, as a function of the comparison, the infection status of the user,
wherein determining further comprises:
receiving training data which contains inputs containing a plurality of user data and infection criterion correlated to outputs containing a plurality of infection statuses;
training an infection status machine learning model with the training data; and
determining, as a function of the infection status machine learning model and the user data, the infection status of the user, wherein the input to the machine learning model is the user data and the output to the machine learning model is the infection status; and
provide the infection status to the user; and
display a recommended consultation on a remote device.

2. The system of claim 1, wherein the computing device is further configured to generate a digital access link that allows access to the computing device from a remote computing device.

3. The system of claim 1, wherein the computing device is further configured to interact with the user through a mobile application.

4. The system of claim 1, wherein the computing device further compares the user data to an infection risk threshold.

5. The system of claim 1, wherein the computing device is further configured to generate a risk profile for a user as a function of the user data.

6. The system of claim 1, wherein the computing device is further configured to communicate with an infection expert database.

7. The system of claim 6, wherein the computing device is further configured to compare the user data with infection expert data of the infection expert database to determine the infection status.

8. The system of claim 1, wherein the computing device is further configured to update the infection status of the user as a function of an infection status of at least another user.

9. The system of claim 1, wherein the computing device is further configured to alert the user of an infection risk as a function of a proximity criterion.

10. The system of claim 1, wherein the computing device is further configured to determine the infection status of the user.

11. A method of informing a user of a COVID-19 infection status using a computing device, comprising:
receiving user data from a user through a diagnostic tool;
comparing the user data to an infection criterion, wherein the infection criterion comprises a weighted criterion configured to be updated as a function of previously generated infection statuses;
determining, as a function of the comparison, the infection status of the user, where determining further comprises:
receiving training data;
training an infection status machine learning model as a function of the training data; and
determining, as a function of the infection status machine learning model and the user data, the infection status of a user; and
providing the infection status to the user; and
display a recommended consultation on a remote device.

12. The method of claim 11, wherein the computing device is further configured to generate a digital access link that allows access to the computing device from a remote computing device.

13. The method of claim 11, wherein the computing device is further configured to interact with the user through a mobile application.

14. The method of claim 11, wherein the computing device further compares the user data to an infection risk threshold.

15. The method of claim 11, wherein the computing device is further configured to generate a risk profile for the user as a function of the user data.

16. The method of claim 11, wherein the computing device is further configured to communicate with an infection expert database.

17. The method of claim 16, wherein the computing device is further configured to compare the user data with infection expert data of the infection expert database to determine the infection status.

18. The method of claim 11, wherein the computing device is further configured to update the infection status of the user as a function of an infection status of at least another user.

19. The method of claim 11, wherein the computing device is further configured to alert the user of an infection risk as a function of a proximity criteria.

20. The method of claim 11 further comprising determining the infection status of the user.
